(19) United States
(12) Patent Application Publication
(10) Pub. No.: US 2026/0256473 A1
(43) Pub. Date: Sep. 3, 2026

(54) IN-VIVO INDWELLING DEVICE

(71) Applicant: KANEKA CORPORATION, Osaka (JP)

(72) Inventor: Hidekazu Nakanishi, Osaka (JP)

(73) Assignee: KANEKA CORPORATION, Osaka (JP)

(21) Appl. No.: 19/652,636

(22) Filed: Apr. 20, 2026

Related U.S. Application Data
(63) Continuation of application No. PCT/JP2024/040047, filed on Nov. 12, 2024.

(30) Foreign Application Priority Data
Dec. 20, 2023 (JP) 2023-214556

(51) Int. Cl. A61B 17/12

(52) U.S. Cl. CPC A61B 17/1215; A61B 17/12113; A61B 17/12154

(57) ABSTRACT

An in-vivo indwelling device is capable of promoting early thrombus formation at a target site such as the inside of an aneurysm and that is unlikely to damage other objects even when it comes into contact with the other objects. The in-vivo indwelling device includes a coil having a longitudinal-axis direction and containing a wire wound around the coil, and a fiber that is disposed in a lumen of the coil. A portion of the fiber extends outward from the coil, and outside the coil, the fiber has a branch point at which the fiber branches.

### CROSS-REFERENCE TO RELATED APPLICATIONS

The present application claims the benefit of Japanese Patent Application No. 2023-214556, filed December 20, 2023. The entire disclosure of Japanese Patent Application No. 2023-214556, filed December 20, 2023, is incorporated herein by reference.

### TECHNICAL FIELD

One or more embodiments of the present invention relate to an in-vivo indwelling device to be indwelled in a body lumen such as a blood vessel.

### BACKGROUND

Intravascular treatment is one of the treatment methods for vascular lesions such as aneurysms of the head and neck, arteriovenous malformations, arteriovenous fistulas, pulmonary vascular malformations, renal vascular malformations, renal arteries aneurysms, and abdominal aneurysms. Embolization is used to prevent rupture of aneurysms such as arterial aneurysms by placing an in-vivo indwelling device having a coil for embolization at a target site such as the inside of an aneurysm to promote thrombosis.

For example, PTL 1 discloses a vasoocclusive device that includes an outer helically wound primary coil having a first end and a second end and defining a lumen between the first end and the second end, and a stretch-resistant member extending through the lumen and fixedly attached to the primary coil at at least two locations. The stretch-resistant member includes a plurality of fibers. PTL 2 discloses a vascular occlusion implant, and the implant includes, in combination, an elongated core member, a first fibrous member attached to the elongated core member, and a second fibrous member attached to the elongated core member. The elongated core member has a proximal end and a distal end. The first fibrous member includes a first polymer material, and the second fibrous member includes a second polymer material different from the first polymer material. PTL 3 discloses an embolic coil that includes an elongated core element formed of a shape memory material processed to form a memorized second coil shape, and an elongated outer element wound around the elongated core element to form a first coil shape of the embolic coil, and includes a plurality of fibers extending from the embolic coil. PTL 4 discloses an occlusion device including a coiled fiber having a coiled configuration and including a first bioabsorbable material composition, and a plurality of intersecting microfibers attached to at least a portion of the coiled fiber, extending radially from an outer diameter portion of the coiled fiber, and including a second bioabsorbable material composition.

### PATENT LITERATURE

PTL 1: Japanese Unexamined Patent Application Publication No. 10-000198

PTL 2: Japanese Unexamined Patent Application Publication (Translation of PCT Application) No. 2002-502659

PTL 3: Japanese Unexamined Patent Application Publication (Translation of PCT Application) No. 2006-528512

PTL 4: Japanese Unexamined Patent Application Publication No. 2022-159143

In-vivo indwelling devices such as those disclosed in PTLs 1 to 4 have room for improvement in terms of early thrombus formation at a target site such as the inside of an aneurysm after placement of the in-vivo indwelling device at the target site. In addition, in-vivo indwelling devices such as those disclosed in PTLs 1 to 4 have a possibility that the distal end or the like of the in-vivo indwelling device may come into contact with a wall of a body lumen such as an inner wall of an aneurysm, thereby damaging the lumen wall when the in-vivo indwelling device is transported to a target site, and thus, there is room for improvement in terms of enhancing safety.

### SUMMARY

One or more embodiments of the present invention have been made in view of the above, and an in-vivo indwelling device capable of promoting thrombus formation at an early stage in a target site such as the inside of an aneurysm and that is less likely to damage other objects even when it comes into contact therewith is provided.

An in-vivo indwelling device according to one or more embodiments of the present invention is as follows.

[1] An in-vivo indwelling device including:

a coil that has a longitudinal-axis direction and around which a wire is wound; and

a fiber that is disposed in a lumen of the coil,

in which the fiber partially extends outward from the coil, and

in which, outside the coil, the fiber has a branch point at which the fiber branches.

[2] The in-vivo indwelling device according to [1], further comprising a fiber bundle including the fiber, a proximal end of the fiber bundle being disposed in a lumen of the coil, and a distal end of the fiber bundle being exposed from the coil.

[3] The in-vivo indwelling device according to [2], in which 30% or more of all fibers constituting the fiber bundle have the branch point.

[4] The in-vivo indwelling device according to [2] or [3], in which, in a state in which the fiber bundle is viewed from a distal end, the fiber bundle has a central region that is a region surrounded by a circle having a diameter of 1/2 of a diameter of a circumscribed circle of the fiber bundle and being centered on a centroid of a lumen of the coil as viewed from a distal end of the coil, and a peripheral region that is a region obtained by removing the central region from a circumscribed circle of the fiber bundle, and

in which the fiber bundle includes a portion in which a fiber density of the fiber bundle in the central region is higher than a fiber density of the fiber bundle in the peripheral region.

[5] The in-vivo indwelling device according to any one of [2] to [4], in which the fiber bundle includes a fixed portion in which a relative position between the fiber bundle and the coil is fixed and a free portion in which a relative position between the fiber bundle and the coil is not fixed, and

in which the branch point located in a portion of the free portion exposed from the coil is greater in number than the branch point located in the fixed portion.

[6] The in-vivo indwelling device according to [5], in which a diameter of a circumscribed circle of the fiber bundle in the free portion in a cross section perpendicular to a longitudinal-axis direction of the coil is larger than an inner diameter of a distal end of the coil.

[7] The in-vivo indwelling device according to any one of [2] to [6], further including a stretch-resistant member disposed in a lumen of the coil,

in which the fiber bundle includes a bundling portion in which proximal end portions of a plurality of the fibers are bundled and fixed, and

in which the bundling portion and the stretch-resistant member are connected.

[8] The in-vivo indwelling device according to [7], in which the bundling portion has a resin tube, and

in which proximal end portions of a plurality of the fibers are disposed in a lumen of the resin tube.

[9] The in-vivo indwelling device according to [5], in which a length of the fixed portion in a longitudinal-axis direction of the coil is longer than an average length of the fiber that is in the free portion and that is exposed from the coil in a longitudinal-axis direction of the coil.

[10] The in-vivo indwelling device according to any one of [1] to [9], in which the coil includes, at a distal end portion of the coil, a reduced-diameter portion where an inner diameter of the coil is reduced.

[11] The in-vivo indwelling device according to [10], in which the reduced-diameter portion includes a proximal reduced-diameter portion including a proximal end of the reduced-diameter portion and a distal reduced-diameter portion located distal to a distal end of the proximal reduced-diameter portion, and

in which an average inner diameter of the proximal reduced-diameter portion is smaller than an average inner diameter of the distal reduced-diameter portion.

[12] The in-vivo indwelling device according to [10] or [11], in which the fiber bundle includes a fixed portion in which a relative position between the fiber bundle and the coil is fixed and a free portion in which a relative position between the fiber bundle and the coil is not fixed, and

in which a length of the fiber that is in the free portion and that is exposed from the coil in a longitudinal-axis direction of the coil is longer than a length of the reduced-diameter portion in a longitudinal-axis direction of the coil.

[13] The in-vivo indwelling device according to any one of [1] to [12], wherein the fiber contains collagen.

According to an in-vivo indwelling device of one or more embodiments of the present invention, specific surface areas of fibers are increased by having fibers that are disposed in a lumen of a coil and that partially extend to the outside of the coil, and by having a branch point at which the fibers branch outside the coil. As a result, thrombus is likely to adhere to fibers that are exposed from the coil, thereby promoting thrombosis, and a distal end portion of the in-vivo indwelling device is covered with the fibers, which makes it unlikely to damage other objects.

### BRIEF DESCRIPTION OF THE DRAWINGS

FIG. 1 is a side view (partial cross-sectional view) along a longitudinal-axis direction of an in-vivo indwelling device according to one or more embodiments of the present invention.

FIG. 2 is an enlarged view of a fiber included in the in-vivo indwelling device illustrated in FIG. 1.

FIG. 3 is a diagram illustrating a fiber bundle of the in-vivo indwelling device illustrated in FIG. 1 as viewed from a distal end.

FIG. 4 is a side view (partial cross-sectional view) along a longitudinal-axis direction of an in-vivo indwelling device according to one or more embodiments of the present invention.

### DETAILED DESCRIPTION

Hereinafter, one or more embodiments of the present invention will be described more specifically on the basis of the following one or more embodiments. However, the present invention is not limited to the following one or more embodiments, and it is of course possible to carry out the present invention with appropriate modifications within the scope that falls within the gist described above and below, and all of them are included in the technical scope of the present invention. In each of the drawings, hatching, the reference signs of components, and the like may sometimes be omitted for convenience. In such cases, reference may be made to the specification or other drawings. Additionally, the dimensions of various components in the drawings may differ from the actual dimensions as priority is given to facilitating understanding of the features of one or more embodiments of the present invention.

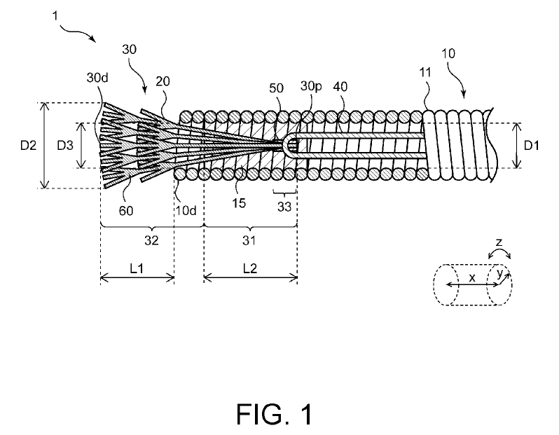

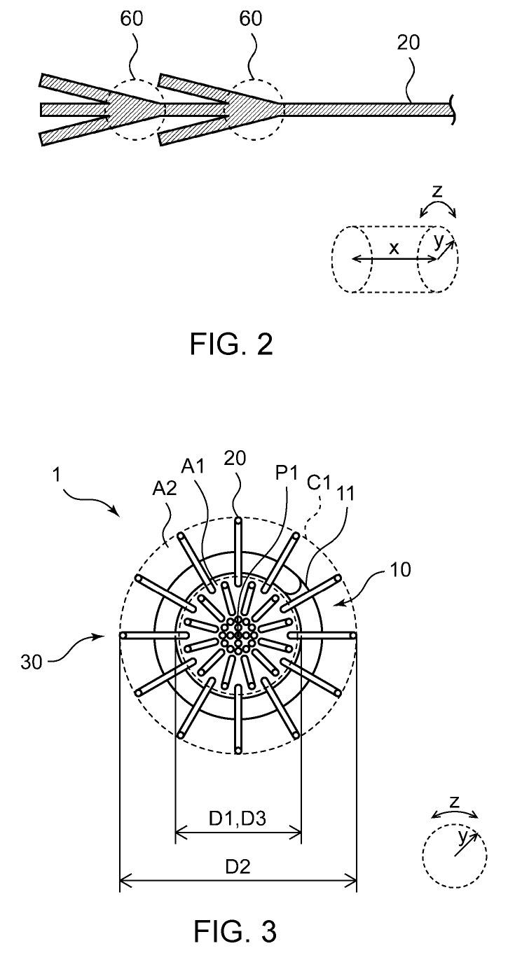

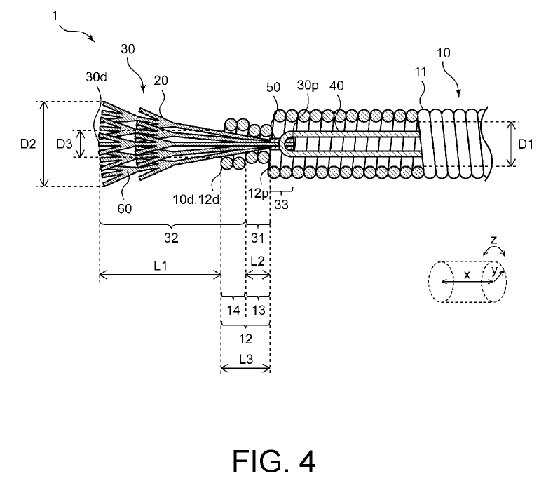

FIG. 1 is a side view (partial cross-sectional view) along a longitudinal-axis direction x of an in-vivo indwelling device 1 according to one or more embodiments of the present invention. FIG. 2 is an enlarged view of a fiber 20 included in the in-vivo indwelling device 1. FIG. 3 is a diagram illustrating a fiber bundle 30 included in the in-vivo indwelling device 1 as viewed from a distal end. FIG. 4 is a side view (partial cross-sectional view) along the longitudinal-axis direction x of the in-vivo indwelling device 1 according to one or more embodiments of the present invention.

As illustrated in FIG. 1 and FIG. 4, the in-vivo indwelling device 1 has a coil 10 around which a wire 11 is wound and which has a longitudinal-axis direction x, and fibers 20 disposed in the lumen of the coil 10.

In one or more embodiments of the present invention, with respect to the longitudinal-axis direction x of the coil 10, the proximal side refers to a side toward an operator, and the distal side refers to the side opposite to the proximal side, that is, the side on which treatment is performed by using the in-vivo indwelling device 1 (lesion side). The longitudinal-axis direction x of the coil 10 may be referred to as an extending direction of the coil 10. The longitudinal-axis direction x of the coil 10 can be rephrased as a proximal-distal direction of the coil 10.

A radial direction y and a circumferential direction z are defined as directions orthogonal to the longitudinal-axis direction x. The radial direction y is a direction perpendicular to the longitudinal-axis direction x and is a direction connecting the centroid of the outer edge of the coil 10 and a point on the outer edge in a cross section perpendicular to the longitudinal-axis direction x. The circumferential direction z is a direction along the outer edge of the coil 10 in a cross section perpendicular to the longitudinal-axis direction x.

The coil 10 is formed by winding the wire 11. In the coil 10, it is preferable that a primary shape be formed by winding the wire 11 and that a secondary shape be formed by winding a coil portion of the primary shape. The primary shape of the coil 10 is also referred to as a primary coil, and may be formed by helically winding the wire 11. The secondary shape of the coil 10 is also referred to as a secondary coil, and may be obtained by further imparting an arc shape, a wave shape, a meandering shape, a zigzag shape, a spiral shape (also referred to as a two-dimensional helical shape or a spiral shape), a ball shape, a box shape, or any other random curved shape without a loop to the primary coil.

The material constituting the wire 11, which forms the coil 10, may have biocompatibility and flexibility. Examples of the material constituting the wire 11 include metals such as platinum, gold, titanium, tungsten and alloys thereof, stainless steel, and combinations thereof. In particular, the material constituting the wire 11 may be a platinum-tungsten alloy. By using a platinum-tungsten alloy as the material constituting the wire 11, the coil 10 has excellent flexibility, and for example, a target site such as the inside of an aneurysm can be more easily filled with the coil 10.

The shape of a cross section of the wire 11 forming the coil 10, the cross section being perpendicular to the longitudinal-axis direction x, may be a circular shape, an oval shape, a polygonal shape, a combination thereof, or the like. Note that the oval shape includes an elliptical shape, an egg shape, and a rectangular shape with rounded corners. The outer diameter of the wire 11 forming the coil 10 may be, for example, 25 μm or more, 30 μm or more, or 35 μm or more, and may be 120 μm or less, 100 μm or less, or 70 μm or less.

The wire 11 forming the coil 10 has a distal end and a proximal end. The wire 11 may be formed of a single linear member from the distal end to the proximal end, or may be formed of a plurality of linear members connected to each other in the longitudinal-axis direction x.

The coil 10 may be a single-layer coil having a single layer or a multi-layer coil having a plurality of layers. In addition, in the longitudinal-axis direction x of the coil 10, a portion of the coil 10 may be formed of a single layer, and the remaining portion of the coil 10 may be formed of a plurality of layers.

The density of the coil 10, that is, the winding interval of the wire 11 forming the coil 10 is not particularly limited, and examples thereof include close winding, open winding, and a combination thereof. Portions of the wire 11 that are adjacent to each other may be in contact with each other in a portion of the coil 10 along the longitudinal-axis direction x, or the adjacent portions of the wire 11 may be in contact with each other throughout the entire length of the coil 10 along the longitudinal-axis direction x. Note that a state in which portions of the wire 11 that are adjacent to each other in the longitudinal-axis direction x of the coil 10 are in contact with each other is referred to as close winding, and a state in which the adjacent portions of the wire 11 are not in contact with each other is referred to as open winding. The state in which portions of the wire 11 that are adjacent to each other in the longitudinal-axis direction x of the coil 10 are not in contact with each other refers to a state in which there is a gap between the adjacent portions of the wire 11 in the longitudinal-axis direction x of the coil 10.

The shape of the cross section of the coil 10 perpendicular to the longitudinal-axis direction x may be a circular shape, an oval shape, a polygonal shape, a combination thereof, or the like. The maximum outer diameter and the minimum outer diameter of the coil 10 can be appropriately selected according to the size of an aneurysm, a procedure, and the like. For example, the minimum outer diameter of the coil 10 may be 150 μm or more, 180 μm or more, or 200 μm or more, and the maximum outer diameter of the coil 10 may be 500 μm or less, 380 μm or less, or 350 μm or less.

As illustrated in FIGS. 1 and 4, the fibers 20 are partially disposed in the lumen of the coil 10. Examples of the material constituting the fibers 20 include collagen, polyester such as polyethylene terephthalate, polypropylene, polyurethane, PLA/PGA polymer, and combinations thereof.

In particular, the fibers 20 may contain collagen. In other words, the in-vivo indwelling device 1 may have collagen fibers. By containing collagen, the fibers 20 can enhance the binding property between the fibers 20 and a thrombus. As a result, a thrombus is easily formed in the in-vivo indwelling device 1, and early thrombus formation can be easily promoted.

The outer diameter of each of the fibers 20 may be 5 μm or more, 10 μm or more, or 15 μm or more. By setting the lower limit of the outer diameter of each of the fibers 20 within the above range, a thrombus can easily adhere to the fibers 20, and formation of a thrombus can be promoted. The outer diameter of each of the fibers 20 may be 150 μm or less, 100 μm or less, or 50 μm or less. By setting the upper limit of the outer diameter of each of the fibers 20 within the above range, the fibers 20 become flexible. Therefore, by covering a distal end portion of the in-vivo indwelling device 1 with the fibers 20, damage to other objects caused by contact of the in-vivo indwelling device 1 with the other objects is less likely to occur.

As illustrated in FIGS. 1 and 4, portions of the fibers 20 extend outside the coil 10. In other words, portions of the fibers 20 are disposed in the lumen of the coil 10, and portions different from the portions disposed in the lumen of the coil 10 extend outside the coil 10 and are exposed.

One end of each of the fibers 20 may be disposed in the lumen of the coil 10, and the other end of each of the fibers 20 may be disposed outside the coil 10. The proximal end of each of the fibers 20 may be disposed in the lumen of the coil 10, and the distal end of each of the fibers 20 may be disposed outside the coil 10. By disposing one end portion of each of the fibers 20 in the lumen of the coil 10 and disposing the other end portion outside the coil 10, the specific surface area of the fibers 20 located outside the coil 10 can be easily increased, and the effect of promoting thrombus formation and the effect of covering the distal end portion of the in-vivo indwelling device 1 with the fibers 20, thereby making it difficult for the in-vivo indwelling device 1 to damage other objects, can be enhanced.

As illustrated in FIGS. 1, 2, and 4, outside the coil 10, the fibers 20 have branch points 60 at which the fibers 20 branch. Since the fibers 20 have the branch points 60, the specific surface area of the fibers 20 exposed from the coil 10 can be increased. As a result, thrombus tends to adhere to the surfaces of the fibers 20, and thrombus formation on the in-vivo indwelling device 1 can be promoted. In addition, the flexibility of the fibers 20 is increased to provide an excellent cushioning property, and the fibers 20 exposed from the coil 10 cover the distal end portion of the in-vivo indwelling device 1, whereby even if the distal end portion of the in-vivo indwelling device 1 comes into contact with a wall of a body lumen such as an inner wall of an aneurysm, the lumen wall is unlikely to be damaged, and the in-vivo indwelling device 1 with high safety can be provided.

Each of the branch points 60 is a point at which a corresponding one of the fibers 20 branches in two or more directions. Even when the fiber 20 is partially split and branched along the extending direction thereof, the fiber 20 can be regarded as having the branch point 60.

The number of the branch points 60 of each of the fibers 20 may be one, or two or more. When each of the fibers 20 has a plurality of branch points 60, the specific surface area of the fibers 20 can be further increased, and the flexibility of the fibers 20 can be further enhanced.

As illustrated in FIG. 1 and FIG. 4, the in-vivo indwelling device 1 includes the fibers 20 and may include a fiber bundle 30 having a proximal end 30p positioned in the lumen of the coil 10 and a distal end 30d exposed from the coil 10. In other words, it is preferable that a portion of the fiber bundle 30 including the proximal end 30p of the fiber bundle 30 be disposed in the lumen of the coil 10 and that a portion of the fiber bundle 30 including the distal end 30d of the fiber bundle 30 be exposed to the outside from the lumen of the coil 10. In addition, it is preferable that the in-vivo indwelling device 1 include the fiber bundle 30 and that the fiber bundle 30 be formed of the plurality of fibers 20 including the fibers 20 having the branch points 60. Since the in-vivo indwelling device 1 includes the fiber bundle 30 including the fibers 20 having the branch points 60, a thrombus is easily formed in the fiber bundle 30, and it is possible to achieve early thrombus formation. In addition, since the in-vivo indwelling device 1 includes the fiber bundle 30, the distal end portion of the in-vivo indwelling device 1 can be easily covered with the fiber bundle 30, and the in-vivo indwelling device 1 that is less likely to damage other objects even when it comes into contact with other objects can be provided.

The fiber bundle 30 may be formed by bundling one end portion of each of the plurality of fibers 20 together or may be formed by folding each of the plurality of fibers 20 in two at a central portion thereof and bundling the folded portions of the plurality of fibers 20 together. In particular, the fiber bundle 30 may be formed by bundling the one end portions of the plurality of fibers 20. By configuring the fiber bundle 30 by bundling the one end portions of the plurality of fibers 20, the plurality of fibers 20 are likely to be firmly fixed to each other, and the fibers 20 are unlikely to fall off from the fiber bundle 30.

The fiber bundle 30 may have five or more fibers 20, ten or more fibers 20, or fifteen or more fibers 20. By setting the lower limit of the number of the fibers 20 included in the fiber bundle 30 within the above range, the distal end 30d of the fiber bundle 30 becomes more likely to expand in the radial direction y. In addition, the fiber bundle 30 may have 500 or less fibers 20, 400 or less fibers 20, or 300 or less fibers 20. By setting the upper limit of the number of the fibers 20 included in the fiber bundle 30 within the above range, the outer diameter at the distal end 30d of the fiber bundle 30 is prevented from becoming excessively large, and the in-vivo indwelling device 1 can be provided with good deliverability.

The fiber bundle 30 may include fibers made of a material other than collagen, or all the fibers 20 may be collagen fibers. In particular, it is preferable that all the fibers 20 constituting the fiber bundle 30 be collagen fibers. When all the fibers 20 constituting the fiber bundle 30 are collagen fibers, the binding property between the fibers 20 and a thrombus is enhanced throughout the fiber bundle 30, and a thrombus is more easily formed.

The fiber bundle 30 may include at least one fiber 20 having the branch point 60, but it is preferable that 30% or more of all the fibers 20 constituting the fiber bundle 30 have the branch points 60. When 30% or more of the fibers 20 constituting the fiber bundle 30 have the branch points 60, the specific surface area of the fibers 20 in the fiber bundle 30 is increased, and the flexibility of the fibers 20 becomes more easily enhanced. As a result, adhesion of a thrombus to the fiber bundle 30 is facilitated, which in turn enhances the effect of promoting thrombus formation, and the cushioning property of the fiber bundle 30 is enhanced, so that damage to other objects due to contact with the in-vivo indwelling device 1 is less likely to occur.

The percentage of the fibers 20 having the branch points 60 among all the fibers 20 included in the fiber bundle 30 may be 50% or more, 70% or more, or 90% or more. By setting the lower limit of the percentage of the fibers 20 having the branch points 60 included in the fiber bundle 30 within the above range, the specific surface area of the fibers 20 constituting the fiber bundle 30 can be increased, and the fibers 20 easily become flexible. Although the upper limit of the percentage of the fibers 20 having the branch points 60 included in the fiber bundle 30 is not particularly limited, it may be, for example, 100% or less. When the percentage of the fibers 20 having the branch points 60 included in the fiber bundle 30 is 100%, all of the fibers 20 included in the fiber bundle 30 have the branch points 60. It is particularly preferable that all the fibers 20 included in the fiber bundle 30 each have at least one branch point 60.

As illustrated in FIG. 3, in a state where the fiber bundle 30 is viewed from the distal end 30d, the fiber bundle 30 has a central region A1 and a peripheral region A2. The central region A1 is a region surrounded by a circle that has a diameter of 1/2 times a diameter D2 of a circumscribed circle C1 of the fiber bundle 30 and that is centered on a centroid P1 of the lumen of the coil 10 as viewed from a distal end 10d of the coil 10. The peripheral region A2 is a region obtained by removing the central region A1 from the circumscribed circle C1 of the fiber bundle 30. It is preferable that the fiber bundle 30 have a portion in which the fiber density of the fiber bundle 30 in the central region A1 is higher than the fiber density of the fiber bundle 30 in the peripheral region A2. The circumscribed circle C1 of the fiber bundle 30 refers to the smallest circumscribed circle including all the fibers 20 constituting the fiber bundle 30 in a state in which the fiber bundle 30 is viewed from the distal end 30d. The centroid P1 of the lumen of the coil 10 refers to the centroid of a shape formed by the lumen of the coil 10 located on the most distal side in a state where the coil 10 is viewed from the distal end 10d. The fiber density of the fiber bundle 30 in the central region A1 is the total area of the fibers 20 per unit area in the central region A1 in a state where the fiber bundle 30 is viewed from the distal end 30d, and is a value obtained by dividing the total area of all the fibers 20 present in the central region A1 in a state where the fiber bundle 30 is viewed from the distal end 30d by the area of the central region A1. The fiber density of the fiber bundle 30 in the peripheral region A2 is the total area of the fibers 20 per unit area in the peripheral region A2 in a state where the fiber bundle 30 is viewed from the distal end 30d, and is a value obtained by dividing the total area of all the fibers 20 present in the peripheral region A2 in a state where the fiber bundle 30 is viewed from the distal end 30d by the area of the peripheral region A2. Since the fiber bundle 30 has a portion in which the fiber density of the fiber bundle 30 in the central region A1 is higher than the fiber density of the fiber bundle 30 in the peripheral region A2, the fiber density in the central region A1 becomes high, and as a result, the flexibility of the entire fiber bundle 30 can be increased. Therefore, during placement of the in-vivo indwelling device 1, when the distal end portion of the in-vivo indwelling device 1 comes into contact with a wall of a body lumen, the pressure is easily absorbed or dispersed by the fiber bundle 30, and the lumen wall can be made less likely to be damaged.

Although not illustrated, in a state where the fiber bundle 30 is viewed from the distal end 30d, it is also preferable that the fiber bundle 30 have a central portion that is a region including the centroid P1 of the lumen of the coil 10 as viewed from the distal end 10d of the coil 10, an intermediate portion that is a region located outside the central portion, and an outer peripheral portion that is a region located outside the central portion and the intermediate portion. It is preferable that the fiber bundle 30 have a portion in which the fiber density of the fiber bundle 30 in each of the central portion and the outer peripheral portion is higher than the fiber density of the fiber bundle 30 in the intermediate portion. Specifically, it is preferable that the fiber density of the fiber bundle 30 be high in the central portion, that the fiber density of the fiber bundle 30 be low in the intermediate portion, and that the fiber density of the fiber bundle 30 be high in the outer peripheral portion. Since the fiber bundle 30 is configured to have a portion in which the fiber density of the fiber bundle 30 in each of the central portion and the outer peripheral portion is higher than the fiber density of the fiber bundle 30 in the intermediate portion, the higher fiber density in each of the central portion and the outer peripheral portion of the fiber bundle 30 enhances the flexibility, and the lower fiber density in the intermediate portion enhances the cushioning property of the entire fiber bundle 30. As a result, the fiber bundle 30 easily absorbs and disperses the load when the distal end portion of the in-vivo indwelling device 1 comes into contact with other objects such as a body lumen wall, so that damage to the other objects is less likely to occur.

As illustrated in FIGS. 1 and 4, the fiber bundle 30 may include a fixed portion 31 where the relative position between the fiber bundle 30 and the coil 10 is fixed and a free portion 32 where the relative position between the fiber bundle 30 and the coil 10 is not fixed. The fixed portion 31 is a portion in which the relative position of the fiber bundle 30 with respect to the coil 10 is fixed and in which the relative position between the fiber bundle 30 and the coil 10 does not change. The free portion 32 is a portion in which the relative position of the fiber bundle 30 with respect to the coil 10 is not fixed and in which the relative position between the fiber bundle 30 and the coil 10 can be changed. As an example of a specific configuration, in the fixed portion 31 of the in-vivo indwelling device 1 illustrated in FIG. 1, the fiber bundle 30 and the coil 10 are bonded to each other by an adhesive 15, and the relative position of the fiber bundle 30 with respect to the coil 10 is fixed. In the fixed portion 31 of the in-vivo indwelling device 1 illustrated in FIG. 4, the coil 10 includes a reduced-diameter portion 12, which will be described later, and the fiber bundle 30 and the coil 10 are in close contact with each other in at least a portion of the reduced-diameter portion 12, whereby the relative position of the fiber bundle 30 with respect to the coil 10 is fixed.

Since the fiber bundle 30 includes the fixed portion 31, the fiber bundle 30 is fixed to the coil 10, and the length of each of the fibers 20 in the free portion 32 can be made constant at the time of delivery of the in-vivo indwelling device 1 or the like. In addition, since the fiber bundle 30 includes the free portion 32, the fibers 20 in the free portion 32 can move relative to the coil 10. Therefore, blood easily passes through the free portion 32 of the in-vivo indwelling device 1 after placement, and a thrombus easily adheres to the free portion 32, so that thrombus formation on the in-vivo indwelling device 1 is promoted.

As illustrated in FIG. 1, the fixed portion 31 may include the proximal end 30p of the fiber bundle 30. When the fixed portion 31 includes the proximal end 30p of the fiber bundle 30, the proximal end portion including the proximal end 30p of the fiber bundle 30 can be easily fixed to the coil 10, and the fibers 20 constituting the fiber bundle 30 can be made less likely to fall off from the coil 10.

The free portion 32 may include the distal end 30d of the fiber bundle 30. In other words, the distal end 30d of the fiber bundle 30 may be located in the free portion 32 of the fiber bundle 30. When the free portion 32 includes the distal end 30d of the fiber bundle 30, the distal end portion including the distal end 30d of the fiber bundle 30 can move with respect to the coil 10. As a result, blood easily enters between the plurality of fibers 20 in the free portion 32, and a thrombus is easily formed in the free portion 32.

The number of the branch points 60 located in the portion of the free portion 32 exposed from the coil 10 may be greater than the number of the branch points 60 located in the fixed portion 31. Also, the number of the branch points 60 located outside the coil 10 may be greater than the number of the branch points 60 located in the lumen of the coil 10. By having the number of the branch points 60 located in the portion of the free portion 32 exposed from the coil 10 be greater than the number of the branch points 60 located in the fixed portion 31, in the portion of the fiber bundle 30 exposed from the coil 10, the fibers 20 can be made flexible and the specific surface area of the fibers 20 can be increased. Therefore, the effect of promoting thrombus formation by the in-vivo indwelling device 1 and the effect of preventing damage to other objects due to contact with the in-vivo indwelling device 1 can be enhanced.

In addition, by having the number of the branch points 60 located in the fixed portion 31 be less than the number of the branch points 60 located in the portion of the free portion 32 exposed from the coil 10, the number of the branch points 60 can be reduced in the portion of the fiber bundle 30 disposed in the lumen of the coil 10. Therefore, when the in-vivo indwelling device 1 passes through a curved blood vessel or the like and the coil 10 is bent, the fibers 20 disposed in the lumen of the coil 10 are less likely to be cut by being sandwiched between portions of the wire 11 constituting the coil 10, and cut fiber pieces can be prevented from being dispersed in the body or the like.

As illustrated in FIGS. 1 and 4, the number of the fibers 20 exposed to the outside of the coil 10 from the distal end 10d of the coil 10 may be greater than the number of the fibers 20 exposed to the outside of the coil 10 from the gaps between portions of the wire 11 constituting the coil 10. Note that FIGS. 1 and 4 illustrate a configuration in which the fibers 20 are not exposed to the outside of the coil 10 from a gap between portions of the wire 11 constituting the coil 10. By having the number of the fibers 20 exposed to the outside of the coil 10 from the distal end 10d of the coil 10 be greater than the number of the fibers 20 exposed to the outside of the coil 10 from the gaps between portions of the wire 11 constituting the coil 10, the number of the fibers 20 exposed to the outside of the coil 10 from a side portion of the coil 10 can be reduced, and the slidability of the outer surface of the in-vivo indwelling device 1 can be improved. As a result, when the in-vivo indwelling device 1 is transported to a target site, the in-vivo indwelling device 1 can be smoothly advanced within a catheter. In addition, when the in-vivo indwelling device 1 passes through a curved blood vessel or the like and the coil 10 is bent, the number of fibers 20 sandwiched between portions of the wire 11 constituting the coil 10 can be reduced, and the fibers 20 can be made less likely to be cut by the wire 11.

In the in-vivo indwelling device 1, it is preferable that the fibers 20 not be exposed to the outside of the coil 10 from a gap between portions of the wire 11 constituting the coil 10. In other words, it is preferable that the in-vivo indwelling device 1 not include the fibers 20 exposed to the outside of the coil 10 from a gap between portions of the wire 11 constituting the coil 10. When the fibers 20 are not exposed to the outside of the coil 10 from the gaps between portions of the wire 11 constituting the coil 10, the slidability of the outer surface of the side portion of the in-vivo indwelling device 1 can be enhanced, and the fibers 20 can be prevented from being sandwiched between portions of the wire 11 constituting the coil 10 and cut when the coil 10 is bent.

As illustrated in FIGS. 1, 3, and 4, the diameter D2 of the circumscribed circle C1 of the fiber bundle 30 in the free portion 32 in a cross section perpendicular to the longitudinal-axis direction x of the coil 10 may be larger than an inner diameter D3 of the distal end 10d of the coil 10. The circumscribed circle C1 of the fiber bundle 30 refers to a circumscribed circle having the largest diameter among circles that are centered on the centroid P1 of the lumen of the coil 10 and that circumscribe the fiber bundle 30 in the free portion 32 in a cross section perpendicular to the longitudinal-axis direction x of the coil 10. When the diameter D2 of the circumscribed circle C1 of the fiber bundle 30 in the free portion 32 is larger than the inner diameter D3 of the distal end 10d of the coil 10, the fibers 20 constituting the fiber bundle 30 expand in the radial direction y in the free portion 32, and blood easily enters between the fibers 20. As a result, a thrombus is easily formed in the fiber bundle 30.

The diameter D2 of the circumscribed circle C1 of the fiber bundle 30 in the free portion 32 in the cross section perpendicular to the longitudinal-axis direction x of the coil 10 may be 1.1 times or more, 1.2 times or more, or 1.3 times or more the inner diameter D3 of the distal end 10d of the coil 10. By setting the lower limit of the ratio between the diameter D2 of the circumscribed circle C1 of the fiber bundle 30 in the free portion 32 and the inner diameter D3 of the distal end 10d of the coil 10 within the above range, the fiber bundle 30 becomes more likely to expand in the radial direction y. The diameter D2 of the circumscribed circle C1 of the fiber bundle 30 in the free portion 32 in the cross section perpendicular to the longitudinal-axis direction x of the coil 10 may be 50 times or less, 40 times or less, or 30 times or less the inner diameter D3 of the distal end 10d of the coil 10. By setting the upper limit of the ratio between the diameter D2 of the circumscribed circle C1 of the fiber bundle 30 in the free portion 32 and the inner diameter D3 of the distal end 10d of the coil 10 within the above range, the fiber bundle 30 is less likely to expand excessively, and the deliverability of the in-vivo indwelling device 1 at the time of delivery of the in-vivo indwelling device 1 can be improved.

As illustrated in FIGS. 1 and 4, the in-vivo indwelling device 1 may further include a stretch-resistant member 40 disposed in the lumen of the coil 10. The stretch-resistant member 40 extends in the longitudinal-axis direction x, and the distal end portion and proximal end portion of the stretch-resistant member 40 can be fixed directly or indirectly to the coil 10. When the in-vivo indwelling device 1 includes the stretch-resistant member 40, the elongation of the coil 10 in the longitudinal-axis direction x can be suppressed.

The stretch-resistant member 40 may be a linear member. The stretch-resistant member 40 may be a single wire or a stranded wire. In addition, the stretch-resistant member 40 may be a single layer or a multilayer body having a plurality of layers. For example, the stretch-resistant member 40 may include an inner layer that is formed of a stranded wire formed of a plurality of linear members and an outer layer that is located outside the inner layer and that is formed of a resin composition. The single stretch-resistant member 40 or a plurality of stretch-resistant members 40 may be disposed in the lumen of the coil 10.

The stretch-resistant member 40 may be made of a resin or a metallic material, and examples thereof include metallic materials such as platinum, gold, rhodium, palladium, silver, titanium, tantalum, tungsten and alloys thereof, and stainless steel, and resin materials such as polyester resins such as polyethylene terephthalate, polyamide resins such as nylon, and polyolefin resins such as polyethylene and polypropylene. When the stretch-resistant member 40 is made of a resin, the flexibility can be increased, and the delivery performance of the in-vivo indwelling device 1 can be improved. In addition, the stretch-resistant member 40 made of a resin does not break due to metal fatigue failure during delivery, and it is also possible to reduce the likelihood that the length of the stretch-resistant member 40 will become insufficient when the coil 10 is arranged inside an aneurysm and that the end portion of the coil 10 will stretch in a straight line and become taut. The stretch-resistant member 40 may be made of a material different from that of the coil 10. Specifically, in the case where the coil 10 is made of a platinum-tungsten alloy, the stretch-resistant member 40 may be made of a polypropylene resin.

The cross-sectional shape in the longitudinal-axis direction x of the linear member constituting the stretch-resistant member 40 may be circular, elliptical, polygonal, or a combination thereof. The outer diameter of the stretch-resistant member 40 may be smaller than the inner diameter of the coil 10. The stretch-resistant member 40 may be disposed in the lumen of the coil 10 in a folded state. Therefore, the outer diameter of the linear member constituting the stretch-resistant member 40 may be smaller than 1/2 of the inner diameter of the coil 10, or equal to or smaller than 1/3 of the inner diameter of the coil 10. In addition, the outer diameter of the linear member constituting the stretch-resistant member 40 may be 1/15 or more of the inner diameter of the coil 10,or 1/10 or more of the inner diameter of the coil 10. By setting the upper limit of the outer diameter of the linear member constituting the stretch-resistant member 40 within the above range, the strength of the stretch-resistant member 40 increases, thereby making it possible to prevent breakage of the stretch-resistant member 40. The outer diameter of the linear member constituting the stretch-resistant member 40 can be, for example, 20 μm or more, or 25 μm or more, or 40 μm or less, or 35 μm or less.

The shape of the stretch-resistant member 40 may be formed in a linear shape, a wave shape, or a spiral shape. In particular, it is more preferable that the shape of the stretch-resistant member 40 be formed in a wave shape. By forming the stretch-resistant member 40 in a wave shape, the length of the stretch-resistant member 40 can be ensured in the lumen of the coil 10, and it is possible to reduce the likelihood that the length of the stretch-resistant member 40 will become insufficient and that the end portion of the coil 10 will stretch in a straight line and become taut.

As illustrated in FIGS. 1 and 4, it is preferable that the in-vivo indwelling device 1 further have the stretch-resistant member 40 disposed in the lumen of the coil 10, that the fiber bundle 30 have a bundling portion 33 in which the proximal end portions of the plurality of fibers 20 are bundled and fixed together, and that the bundling portion 33 and the stretch-resistant member 40 be connected. With the configuration in which the bundling portion 33 of the fiber bundle 30 and the stretch-resistant member 40 are connected to each other, the position of the fiber bundle 30 with respect to the coil 10 becomes more easily fixed, and the length of the free portion 32 of the fiber bundle 30 becomes less likely to change due to delivery of the in-vivo indwelling device 1. As a result, the effect of promoting thrombus formation by the in-vivo indwelling device 1 and the effect of making a wall of a body lumen or the like less likely to be damaged can be more stably exhibited.

As illustrated in FIGS. 1 and 4, the bundling portion 33 of the fiber bundle 30 may include a resin tube 50, and the proximal end portions of the plurality of fibers 20 may be disposed in the lumen of the resin tube 50. By disposing the proximal end portions of the plurality of fibers 20 constituting the fiber bundle 30 in the lumen of the resin tube 50, the proximal end portions of the plurality of fibers 20 can be protected by the resin tube 50. Therefore, when the coil 10 is bent by a curved blood vessel or the like, the proximal end portions of the fibers 20 and the coil 10 can be prevented from coming into contact with each other, and the fibers 20 can be made less likely to be broken.

As a material constituting the resin tube 50, for example, a polyamide-based resin such as nylon, a polyether polyamide-based resin, a polyimide-based resin, a polyester-based resin such as polyethylene terephthalate (PET), a polyurethane-based resin, a polyolefin-based resin such as polyethylene or polypropylene, a fluorine-based resin such as polytetrafluoroethylene (PTFE), perfluoroalkoxyalkane (PFA), or an ethylene tetrafluoroethylene copolymer (ETFE), a thermoplastic resin such as a polyvinyl chloride-based resin or a silicone-based resin, natural rubber, or the like may be used. These may be used alone or in combination of two or more. Among these, the material constituting the resin tube 50 may be a fluorine-based resin, or polytetrafluoroethylene. When the material constituting the resin tube 50 is a fluorine-based resin, the lubricity of the inner surface and the outer surface of the resin tube 50 is improved. Therefore, it becomes easier to insert the plurality of fibers 20 into the lumen of the resin tube 50 and to dispose the fiber bundle 30 in the lumen of the coil 10 after disposing the proximal end portions of the plurality of fibers 20 in the lumen of the resin tube 50.

Examples of fixing the proximal end portions of the plurality of fibers 20 by the resin tube 50 include: forming the resin tube 50 from a material that shrinks upon heating, arranging the proximal end portions of the plurality of fibers 20 in the lumen of the resin tube 50, and then heating the resin tube 50 to fix the proximal end portions of the plurality of fibers 20; arranging the proximal end portions of the plurality of fibers 20 in the lumen of the resin tube 50 and then pouring an adhesive into the lumen of the resin tube 50 to fix the proximal end portions of the plurality of fibers 20; and arranging the proximal end portions of the plurality of fibers 20 in the lumen of the resin tube 50 and then fixing the proximal end portions of the plurality of fibers 20 by welding. In particular, it is preferable to fix the proximal end portions of the plurality of fibers 20 by heating the resin tube 50 to cause the resin tube 50 to shrink. By heating the resin tube 50 to cause the resin tube 50 to shrink and thereby fixing the proximal end portions of the plurality of fibers 20, the plurality of fibers 20 can be fixed easily and firmly.

As illustrated in FIG. 1, a length L2 of the fixed portion 31 in the longitudinal-axis direction x of the coil 10 may be longer than the average length of the fibers 20 in the free portion 32 that are exposed from the coil 10 in the longitudinal-axis direction x of the coil 10. In other words, the length L2 of the fixed portion 31 in the longitudinal-axis direction x of the coil 10 may be longer than the average value of lengths L1 of the fibers 20 in the free portion 32 that are exposed from the coil 10 in the longitudinal-axis direction x of the coil 10. By making the length L2 of the fixed portion 31 longer than the average length of the fibers 20 in the free portion 32 that are exposed from the coil 10, the proximal end portion of the fiber bundle 30 becomes more easily supported by the coil 10. Therefore, outside the coil 10, the distal end portions of the fibers 20 are less likely to deflect significantly, and a thrombus is more likely to be formed in a portion of the fiber bundle 30.

The length L2 of the fixed portion 31 in the longitudinal-axis direction x of the coil 10 may be 1.1 times or more, 1.2 times or more, or 1.3 times or more the average value of the lengths L1 of the fibers 20 in the free portion 32 that are exposed from the coil 10 in the longitudinal-axis direction x of the coil 10. By setting the lower limit of the ratio between the length L2 of the fixed portion 31 and the average length of the fibers 20 in the free portion 32 that are exposed from the coil 10 within the above range, the proximal end portion of the fiber bundle 30 is supported by the coil 10, and the distal end portion of the fiber bundle 30 can be made less likely to deflect significantly. In addition, the length L2 of the fixed portion 31 in the longitudinal-axis direction x of the coil 10 may be 3.0 times or less, 2.5 times or less, or 2.0 times or less the average value of the lengths L1 of the fibers 20 in the free portion 32 that are exposed from the coil 10 in the longitudinal-axis direction x of the coil 10. By setting the upper limit of the ratio between the length L2 of the fixed portion 31 and the average length of each of the fibers 20 in the free portion 32 that are exposed from the coil 10 within the above range, the length of each of the fibers 20 exposed from the distal end 10d of the coil 10 can be ensured, and the effect of promoting thrombus formation by the fiber bundle 30 and the effect of preventing damage to other objects by covering the distal end portion of the in-vivo indwelling device 1 with the fibers 20 can be more easily exhibited.

As illustrated in FIG. 4, the coil 10 may include, at the distal end portion of the coil 10, the reduced-diameter portion 12 where the inner diameter D1 of the coil 10 is reduced. When the coil 10 includes the reduced-diameter portion 12, the inner surface of the coil 10 and the fiber bundle 30 come into contact with each other in the reduced-diameter portion 12, and the proximal end portion of the fiber bundle 30 becomes more easily supported by the coil 10. Therefore, on the distal side with respect to the reduced-diameter portion 12, the plurality of fibers 20 constituting the fiber bundle 30 become less likely to deflect significantly, and the fiber bundle 30 becomes more likely to expand in the radial direction y.

The reduced-diameter portion 12 may be located at the distal end portion of the coil 10, and the reduced-diameter portion 12 may be located at the distal end 10d of the coil 10, or the distal end 12d of the reduced-diameter portion 12 may be located closer to the proximal side than the distal end 10d of the coil 10. In particular, the reduced-diameter portion 12 may be located at the distal end 10d of the coil 10. The reduced-diameter portion 12 being located at the distal end 10d of the coil 10 refers to a configuration in which the position of the distal end 12d of the reduced-diameter portion 12 and the position of the distal end 10d of the coil 10 coincide with each other. When the reduced-diameter portion 12 is located at the distal end 10d of the coil 10, the proximal end portion of the fiber bundle 30 is supported by the distal end 10d of the coil 10, and the plurality of fibers 20 constituting the fiber bundle 30 become even less likely to deflect.

The minimum inner diameter of the coil 10 in the reduced-diameter portion 12 may be 90% or less, 80% or less, or 70% or less of the maximum inner diameter of the coil 10. By setting the upper limit of the ratio between the minimum inner diameter of the coil 10 in the reduced-diameter portion 12 and the maximum inner diameter of the coil 10 within the above range, the inner surface of the coil 10 and the fiber bundle 30 become more likely to come into contact with each other in the reduced-diameter portion 12, and the fiber bundle 30 becomes more easily supported by the reduced-diameter portion 12. In addition, the minimum inner diameter of the coil 10 in the reduced-diameter portion 12 may be 15% or more, 20% or more, or 25% or more of the maximum inner diameter of the coil 10. By setting the lower limit of the ratio between the minimum inner diameter of the coil 10 in the reduced-diameter portion 12 and the maximum inner diameter of the coil 10 within the above range, the space in the lumen of the coil 10 in the reduced-diameter portion 12 can be ensured, and the number of the fibers 20 constituting the fiber bundle 30 can be increased or the fiber diameter can be increased.

As illustrated in FIG. 4, the reduced-diameter portion 12 includes a proximal reduced-diameter portion 13 including the proximal end 12p of the reduced-diameter portion 12 and a distal reduced-diameter portion 14 located on the distal side of the distal end of the proximal reduced-diameter portion 13, and the average inner diameter of the proximal reduced-diameter portion 13 may be smaller than the average inner diameter of the distal reduced-diameter portion 14. When the reduced-diameter portion 12 includes the proximal reduced-diameter portion 13 and the distal reduced-diameter portion 14 and the average inner diameter of the proximal reduced-diameter portion 13 is smaller than the average inner diameter of the distal reduced-diameter portion 14, in the reduced-diameter portion 12, the inner diameter of the coil 10 increases toward the distal side, and the inner surface of the coil 10 and the fiber bundle 30 come into contact with each other, so that the fiber bundle 30 becomes more likely to expand in the radial direction y. As a result, blood becomes more likely to enter between the plurality of fibers 20 constituting the fiber bundle 30, and a thrombus becomes more likely to be formed in a portion of the fiber bundle 30.

The average inner diameter of the proximal reduced-diameter portion 13 may be 95% or less, 90% or less, or 85% or less of the average inner diameter of the distal reduced-diameter portion 14. In addition, the average inner diameter of the proximal reduced-diameter portion 13 may be 35% or more, 40% or more, or 45% or more of the average inner diameter of the distal reduced-diameter portion 14. By setting the upper limit and the lower limit of the ratio between the average inner diameter of the proximal reduced-diameter portion 13 and the average inner diameter of the distal reduced-diameter portion 14 within the above ranges, the difference between the average inner diameter of the proximal reduced-diameter portion 13 and the average inner diameter of the distal reduced-diameter portion 14 becomes more likely to be appropriate, and the plurality of fibers 20 constituting the fiber bundle 30 become more likely to expand.

Although not illustrated, the reduced-diameter portion 12 may further include a portion that is different from the proximal reduced-diameter portion 13 and from the distal reduced-diameter portion 14. Specifically, for example, a portion having an inner diameter or the like different from those of the proximal reduced-diameter portion 13 and the distal reduced-diameter portion 14 may be provided on the proximal side of the proximal reduced-diameter portion 13, on the distal side of the proximal reduced-diameter portion 13 and on the proximal side of the distal reduced-diameter portion 14, or on the distal side of the distal reduced-diameter portion 14.

As illustrated in FIG. 4, the lengths L1 of the fibers 20 in the free portion 32 that are exposed from the coil 10 in the longitudinal-axis direction x of the coil 10 may be longer than a length L3 of the reduced-diameter portion 12 in the longitudinal-axis direction x of the coil 10. By making each of the lengths L1 of the fibers 20 in the free portion 32 that are exposed from the coil 10 longer than the length L3 of the reduced-diameter portion 12, the fiber bundle 30 becomes more likely to expand widely on the distal side with respect to the distal end 10d of the coil 10. Therefore, blood becomes more likely to enter between the plurality of fibers 20 constituting the fiber bundle 30, and thrombus formation can be promoted.

The lengths L1 of the fibers 20 in the free portion 32 that are exposed from the coil 10 in the longitudinal-axis direction x of the coil 10 may be 1.1 times or more, 1.2 times or more, or 1.3 times or more the length L3 of the reduced-diameter portion 12 in the longitudinal-axis direction x of the coil 10. By setting the lower limit of the ratio between each of the lengths L1 of the fibers 20 in the free portion 32 that are exposed from the coil 10 and the length L3 of the reduced-diameter portion 12 within the above range, the length L1 of each of the fibers 20 exposed from the coil 10 becomes more likely to be sufficient, and the effect of promoting thrombus formation by the fiber bundle 30, the effect of covering the distal end portion of the coil 10 with the fiber bundle 30 to make it less likely to damage a wall of a body lumen or the like, and the effect of suppressing the movement of the in-vivo indwelling device 1 by the entanglement of the plurality of fibers 20 can be more easily achieved. In addition, the lengths L1 of the fibers 20 in the free portion 32 that are exposed from the coil 10 in the longitudinal-axis direction x of the coil 10 may be 3.0 times or less, 2.5 times or less, or 2.0 times or less the length L3 of the reduced-diameter portion 12 in the longitudinal-axis direction x of the coil 10. By setting the upper limit of the ratio between each of the lengths L1 of the fibers 20 in the free portion 32 that are exposed from the coil 10 and the length L3 of the reduced-diameter portion 12 within the above range, the proximal end portion of the fiber bundle 30 becomes more easily supported by the reduced-diameter portion 12, and the fibers 20 exposed from the coil 10 become less likely to deflect significantly.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### REFERENCE SIGNS LIST

1 in-vivo indwelling device

10 coil

10d distal end of coil

11 wire

12 reduced-diameter portion

12d distal end of reduced-diameter portion

12p proximal end of reduced-diameter portion

13 proximal reduced-diameter portion

14 distal reduced-diameter portion

15 adhesive

20 fiber

30 fiber bundle

30d distal end of fiber bundle

30p proximal end of fiber bundle

31 fixed portion

32 free portion

33 bundling portion

40 stretch-resistant member

50 resin tube

60 branch point

C1 circumscribed circle of fiber bundle in free portion

P1 centroid of lumen of coil viewed from distal portion of coil

A1 central region

A2 peripheral region

L1 length of fiber exposed from coil

L2 length of fixed portion

L3 length of reduced-diameter portion

D1 inner diameter of coil

D2 diameter of circumscribed circle of fiber bundle in free portion

D3 inner diameter of distal end of coil

x longitudinal-axis direction

y radial direction

z circumferential direction

1. An in-vivo indwelling device comprising:
a coil having a longitudinal-axis direction and containing a wire wound around the coil; and
a fiber disposed in a lumen of the coil,
wherein the fiber partially extends outward from the coil, and
wherein, outside the coil, the fiber has a branch point at which the fiber branches.

2. The in-vivo indwelling device according to claim 1, comprising a fiber bundle including the fiber, wherein the fiber bundle has a proximal end disposed in the lumen of the coil and a distal end exposed from the coil.

3. The in-vivo indwelling device according to claim 2, wherein 30% or more of all fibers constituting the fiber bundle have the branch point.

4. The in-vivo indwelling device according to claim 2,
wherein, when the fiber bundle is viewed from a distal end, the fiber bundle has:
a central region surrounded by a circle having a diameter of 1/2 of a diameter of a circumscribed circle of the fiber bundle, wherein the central region is centered on a centroid of the lumen of the coil as viewed from a distal end of the coil; and
a peripheral region that is a region excluding the central region from the circumscribed circle of the fiber bundle, and
wherein the fiber bundle includes a portion in which a fiber density of the fiber bundle in the central region is higher than a fiber density of the fiber bundle in the peripheral region.

5. The in-vivo indwelling device according to claim 2,
wherein the fiber bundle includes:
a fixed portion in which a relative position between the fiber bundle and the coil is fixed; and
a free portion in which a relative position between the fiber bundle and the coil is not fixed, and
wherein a number of branch points located in a portion of the free portion exposed from the coil is greater than a number of branch points located in the fixed portion.

6. The in-vivo indwelling device according to claim 5, wherein a diameter of a circumscribed circle of the fiber bundle in the free portion in a cross section perpendicular to the longitudinal-axis direction of the coil is larger than an inner diameter of a distal end of the coil.

7. The in-vivo indwelling device according to claim 2, further comprising a stretch-resistant member disposed in the lumen of the coil,
wherein the fiber bundle includes a bundling portion in which proximal end portions of a plurality of fibers are bundled and fixed, and
wherein the bundling portion and the stretch-resistant member are connected.

8. The in-vivo indwelling device according to claim 7,
wherein the bundling portion has a resin tube, and
wherein the proximal end portions of the plurality of fibers are disposed in a lumen of the resin tube.

9. The in-vivo indwelling device according to claim 5, wherein a length of the fixed portion in the longitudinal-axis direction of the coil is longer than an average length of the fiber in the free portion and exposed from the coil in the longitudinal-axis direction of the coil.

10. The in-vivo indwelling device according to claim 1, wherein the coil includes, at a distal end portion of the coil, a reduced-diameter portion where an inner diameter of the coil is reduced.

11. The in-vivo indwelling device according to claim 10,
wherein the reduced-diameter portion includes:
a proximal reduced-diameter portion including a proximal end of the reduced-diameter portion; and
a distal reduced-diameter portion located distal to a distal end of the proximal reduced-diameter portion, and
wherein an average inner diameter of the proximal reduced-diameter portion is smaller than an average inner diameter of the distal reduced-diameter portion.

12. The in-vivo indwelling device according to claim 10,
wherein a fiber bundle includes:
a fixed portion in which a relative position between the fiber bundle and the coil is fixed; and
a free portion in which the relative position between the fiber bundle and the coil is not fixed, and
wherein a length of the fiber in the free portion and exposed from the coil in the longitudinal-axis direction of the coil is longer than a length of the reduced-diameter portion in the longitudinal-axis direction of the coil.

13. The in-vivo indwelling device according to claim 1, wherein the fiber contains collagen.
